(19) United States
(12) Patent Application Publication
(10) Pub. No.: US 2025/0288717 A1
(43) Pub. Date: Sep. 18, 2025

(54) METHOD FOR REDUCING IMPERFECTIONS OF GLUTEUS

(71) Applicant: Iani Silveira, Miami, FL (US)

(72) Inventor: Iani Silveira, Miami, FL (US)

(21) Appl. No.: 19/221,483

(22) Filed: May 28, 2025

Related U.S. Application Data
(63) Continuation of application No. 17/830,804, filed on Jun. 2, 2022, now abandoned.

(51) Int. Cl. A61L 27/12; A61B 17/00; A61M 3/02; A61M 5/32

(52) U.S. Cl. CPC A61L 27/12; A61M 5/3294; A61B 2017/00747; A61L 2400/06; A61L 2430/30; A61L 2430/34; A61M 3/0279

(57) ABSTRACT

A method for reducing imperfection of gluteus including hyper diluting Radiesse® filler with a saline solution, mixing the Radiesse® filler with said saline solution to obtain a homogenous mixture, introducing a cannula into a subdermal area of a patient gluteus and administering the homogenous mixture into the subdermal area of a patient in 5-10 ml aliquots 2 cm squared retrograding surrounding an initial injection area. The homogenous mixture has a mix ratio of five parts of saline solution for each part of Radiesse® filler. The cannula being introduced into the subdermal area through an entry point made by an 18 mm needle. The initial injection area is defined by an initial injection of 5 ml of the homogenous mixture into the subdermal area of the patient's gluteus. The present method is used to increase the volume, reshape and reduce imperfections of the patient's gluteus.

### II. BACKGROUND OF THE INVENTION

### 1. Field of the Invention

The present invention relates to a method for reducing imperfections of gluteus and, more particularly, to a method for reducing imperfections of gluteus that enhances the volume of localized gluteal regions of a patient's gluteus to reduce imperfections and increase the volume of the patient's gluteus.

### 2. Description of the Related Art

Several designs for methods for reducing imperfections of gluteus have been designed in the past. None of them, however, include a composition of calcium hydroxylapatite hyper diluted with a saline solution to be injected in a subdermal area of a patient's gluteus to increase and define patient's gluteus in a noninvasive procedure.

Applicant believes that a related reference corresponds to U.S. Pat. No. 11,000,626 issued for a dermal filler compositions including antioxidants. Applicant believes that another related reference corresponds to U.S. Pat. No. 9,295,691 issued for a method for repairing or replacing damaged tissue. None of these references, however, teach of a method for reducing imperfections of gluteus continuously laying 5 to 10 ml of homogenized solution of hyper diluted calcium hydroxylapatite until 2 cm from injection point.

Other documents describing the closest subject matter provide for a number of more or less complicated features that fail to solve the problem in an efficient and economical way. None of these patents suggest the novel features of the present invention.

### III. SUMMARY OF THE INVENTION

It is one of the objects of the present invention to provide a method for reducing imperfections of gluteus that includes an injectable homogeneous composition Radiesse® filler diluted with a saline solution, the Radiesse® filler being hyper diluted calcium hydroxylapatite.

It is another object of this invention to provide a method for reducing imperfections of gluteus that uses square retrograding for injecting the Homogenized solution of hyper diluted calcium hydroxylapatite.

It is another object of the present invention to provide a method for reducing imperfections of gluteus that uses an 18 gauge to make an entry point for a cannula to enter the subdermal area.

It is still another object of the present invention to provide a method for reducing imperfections of gluteus which increase and reshape the patient's gluteus using a noninvasive procedure which avoids a lengthy recovery.

It is yet another object of this invention to provide such a device that is inexpensive to implement and maintain while retaining its effectiveness.

Further objects of the invention will be brought out in the following part of the specification, wherein detailed description is for the purpose of fully disclosing the invention without placing limitations thereon.

### IV. BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 represents a flow chart for a method 10 containing a first step 20, a second step 40, a third step 60 and a fourth step 80 in accordance with an embodiment of the present invention.

### V. DETAILED DESCRIPTION OF THE EMBODIMENTS OF THE INVENTION

Referring now to the drawings, where the present invention is generally referred to with numeral 10, it can be observed that it basically includes a first step 20, a second step 40, a third step 60 and a fourth step 80. It should be understood there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

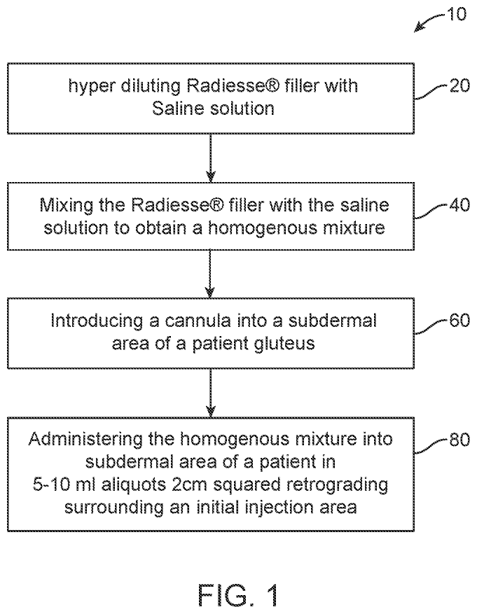

Best illustrated in FIG. 1 first step 20 may include hyper diluting the Radiesse® filler with a saline solution. Wherein the Radiesse® filler is an injectable cosmetic skin treatment used to increase the volume of subdermal areas of a patient to reshape, lift and increase the patient's gluteus. The Radiesse® filler is commonly used for dermal filling in patients. Radiesse® filler is a sterile, latex-free, non-pyrogenic, semi-solid, cohesive subdermal, injectable implant whose principal component is synthetic calcium hydroxylapatite suspended in a gel carrier of sterile water for injection, glycerin, and sodium carboxymethylcellulose. The saline solution may include intravenous fluids such as a saline solution that includes 0.9% sodium chloride. In a preferred embodiment the Radiesse® filler may be hyper diluted in a ratio mix of five parts of the saline solution to one part of calcium hydroxylapatite.

The second step 40 may be mixing Radiesse® filler with the saline solution to obtain a homogenous mixture. The Radiesse® filler and the saline solution may be mixed in a ration mix of one to five. The homogenous mixture may be achieved by using a luer lock. The Radiesse® filler and the saline solution may be mixed 20 times back and forth using the luer lock obtaining a homogenous mixture is beneficial as it prevents clumping or graininess of the solution. Clumping or graininess of the solution before administering subdermally may have unintended or adverse effects on a patient's skin. Adverse effects may include swelling, itching, pain, bruising, redness, or lumps.

The third step 60 may include introducing a cannula into a subdermal area of a patient gluteus. An eighteen-gauge needle may be used to make an entry point for the cannula. The cannula may be inserted into the entry point a maneuvered along the natural planes of the skin tissue. It may be suitable to use lidocaine and epinephrine to numb the gluteal region of the patient before introducing the cannula or the needle into a subdermal area of the patient gluteus. In a preferred embodiment the cannula may have a needle and an injection port. The cannula may be a 90 mm cannula. A 10 ml syringe may be inserted in the injection port of the cannula. The 10 ml syringe may include the homogenous mixture of Radiesse® filler with the saline solution in a ratio mix of one to five.

The fourth step 80 may include administering the homogeneous mixture of Radiesse® filler with the saline solution into a subdermal area of a patient in 5-10 ml aliquots 2 cm square retrograding surrounding an initial injection area. It may be preferable for the homogenous mixture to be administered to a patient by board certified medical professionals. Wherein the board-certified medical professionals may include but are not limited to physicians, physician assistants, advanced nurse practitioners, Doctor of Medicine, or Doctor of Osteopathic Medicine. It may be suitable for the homogenous mixture to be dispensed via a needle. The homogenous mixture may be administered in dermal areas that lack volume. In the current embodiment the homogenous mixture may be injected subdermally into a gluteus of a patient. The homogenous mixture may further be administered to correct dermal areas with cellulite, divots, and or stretch marks. The homogeneous mixture may also be administered to reshape and increase volume of the patient's gluteus.

In a preferred embodiment an initial injection of 5 ml of homogenous mixture may be administered subdermally into the subdermal area of the patient's gluteus. The initial injection may thereby define an initial injection site. 5-10 ml aliquots 2 cm squared may be injected retrograding and placing more product for volume increase surrounding the initial injection site. Thereby allowing for a more natural appearing increased volume gradient surrounding the initial injection site. The retrograde injection is known in the prior art as being a technique wherein the needle is fully advanced without injection, and injection commences as the needle is withdrawn, filling the tract with product. The patient may refrain from strenuous activity, heat exposure, or flattering of the product after 48 hours after administering the homogeneous mixture of Radiesse® filler with the saline solution. The present method and invention may allow for a more pain free experience for the patient and by extension a much faster recovery time compared to invasive procedures.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

1. A method for reducing imperfections of gluteus, comprising:
a) hyper diluting a filler with a saline solution, wherein the filler is an injectable composition comprising synthetic calcium hydroxylapatite microspheres suspended in an aqueous sodium carboxymethylcellulose gel carrier;
b) mixing the filler with the saline solution to obtain a homogenous mixture;
c) introducing a 90 mm cannula into a subdermal area of a patient gluteus, wherein the cannula is introduced through the subdermal area through an entry point, the entry point is made by means of an eighteen-gauge needle, wherein the 90 mm cannula is coupled to a 10 ml syringe the syringe contains the homogenous mixture; and
d) administering the homogenous mixture into the subdermal area of the healthy tissue of the gluteus of the patient in 5-10 ml aliquots 2 cm squared retrograding surrounding an initial injection area, wherein the homogeneous mixture has a mix ratio of five parts of saline solution for each part of the filler, and wherein a volume of the gluteus is increased as a result of the administering the homogenous mixture into the gluteus.

2. The method for reducing imperfections of gluteus set forth in claim 1, wherein the saline solution includes 0.9% sodium chloride.

3. The method for reducing imperfections of gluteus set forth in claim 1, wherein the filler and the saline solution are mixed by means of a luer lock.

4. The method for reducing imperfections of gluteus set forth in claim 3, wherein the homogenous mixture is achieved by mixing the filler and the saline solution by means of the luer lock 20 times back and forth.

5. A method for reducing imperfections of gluteus, consisting of:
a) hyper diluting a filler with a saline solution, wherein the saline solution includes 0.9% of sodium chloride, wherein the filler is an injectable composition comprising synthetic calcium hydroxylapatite microspheres suspended in an aqueous sodium carboxymethylcellulose gel carrier;
b) mixing the filler with the saline solution to obtain a homogenous mixture, wherein the homogeneous mixture has a mix ratio of five parts of saline solution for each part of the filler, the homogenous mixture is achieved by mixing the filler and the saline solution by means of the luer lock 20 times back and forth;
c) introducing a cannula into a subdermal area of a patient gluteus, wherein the cannula is introduced into the subdermal area of the gluteus of the patient through an entry point, the entry point is made by means of a needle, the needle is an eighteen gauge needle, the cannula is a 90 mm cannula, wherein a 10 ml syringe is coupled to the cannula, the syringe contains the homogenous mixture; and
d) administering the homogenous mixture into the subdermal area of the healthy tissue of the gluteus of the patient in 5-10 ml aliquots 2 cm squared retrograding surrounding an initial injection area, wherein the initial injection area is defined by an initial injection of 5 ml of the homogenous mixture in the subdermal area of the gluteus of the patient, and wherein a volume of the gluteus is increased as a result of the administering the homogenous mixture into the gluteus.
